(19) United States
(12) Patent Application Publication
(10) Pub. No.: US 2025/0380856 A1
(43) Pub. Date: Dec. 18, 2025

(54) IMAGING GUIDEWIRE SYSTEM AND METHOD OF USE

(71) Applicants: Csaba TRUCKAI, San Jose, CA (US); Akos TOTH, San Jose, CA (US)

(72) Inventors: Csaba TRUCKAI, San Jose, CA (US); Akos TOTH, San Jose, CA (US)

(21) Appl. No.: 19/242,017

(22) Filed: Jun. 18, 2025

Related U.S. Application Data
(60) Provisional application No. 63/661,249, filed on Jun. 18, 2024.

(51) Int. Cl. A61B 1/005; A61B 1/00; A61B 1/05; A61B 1/06; A61B 1/32

(52) U.S. Cl. CPC A61B 1/0051; A61B 1/00119; A61B 1/00124; A61B 1/00128; A61B 1/00135; A61B 1/00165; A61B 1/05; A61B 1/0676; A61B 1/0684; A61B 1/32; A61B 2560/0462

(57) ABSTRACT

Imaging guidewires and dilators for providing intrauterine access and methods of operation thereof.

### CROSS-REFERENCE TO RELATED APPLICATIONS

This application is a non-provisional of U.S. Provisional application No. 63/661,249 filed Jun. 18, 2024, the entirety of which is incorporated by reference.

### FIELD OF THE INVENTION

This disclosure relates generally to the field of gynecology and, more specifically, to an imaging guidewire and dilator for providing intrauterine access and methods of operation thereof.

### BACKGROUND OF THE INVENTION

Minimally invasive gynecological procedures that require intrauterine access often require effective cervical dilation. Such procedures include diagnostic hysteroscopy, polyp and fibroid resection, placement of intrauterine devices (IUDs), and endometrial ablation.

The cervix, the lower portion of the uterus, acts as a barrier to the uterine cavity. Current methods for cervical dilation include mechanical dilators and laminaria sticks. However, these methods have limitations, including the potential for cervical laceration or perforation. Currently, a standard method for cervical dilation relies on mechanical cervical dilators. These dilators are inserted in a sequential manner, progressively increasing their diameter to achieve the necessary dilation.

Therefore, there is a significant unmet clinical need for improved methods of cervical dilation in gynecology. An ideal method would minimize the risk of cervical injuries, be faster to perform, and effectively achieve intrauterine access in a wider range of patients, including those with challenging cervical anatomy.

### SUMMARY OF THE INVENTION

The present invention addresses this critical need by providing a novel method for cervical dilation that overcomes the limitations of existing techniques, leading to a safer, more efficient, and patient-centered approach to gynecological procedures. This method offers several key advantages. First, the system allows for enhanced visualization using a very small diameter (1 mm to 2.5 mm) flexible optical guidewire configured with an image sensor and LED light source to provide direct visualization of the cervical canal during insertion. This real-time visualization greatly reduces the risk of inadvertent injury and allows for the flexible guidewire tip to naturally follow the path of least resistance through the patient's cervical canal into the uterine cavity. The optical guidewire's small diameter minimizes initial cervical and practically eliminates the risk of cervical lacerations or perforation compared to traditional mechanical dilators. Following visualization with the guidewire, a cervical dilator or sequential dilators can be introduced directly over the guidewire assembly. This ensures the dilator follows the correct path through the cervical canal, enabling controlled and predictable dilation to the desired diameter. The combined use of the guidewire for visualization and the dilator for dilation streamlines the procedure and reduces the overall operative time.

The present disclosure includes improved intrauterine access systems. For example such a system can include a handle; an elongate optical guide member extending about an axis with a flexible distal tip portion carrying an image sensor and a light emitter; a first connector with electrical contacts at a proximal end of the elongate optical guide member for coupling to a cooperating second connector in the handle; an elongate introducer sleeve with an interior passageway dimensioned for receiving the elongate optical guide member; and a hub at the proximal end of the elongate introducer sleeve with a fitting adapted for coupling to a fluid source for delivering a fluid flow into the interior passageway of the elongate introducer sleeve. Variations include the elongate optical guide member having a maximum cross-sectional dimension of 2.5 mm or less.

Variations of the system can include flexible distal tip portions having an axial length of at least 5 mm or at least 10 mm.

The light emitter can comprise at least one LED. Alternatively, or in combination, the light emitter can comprise a fiber optic bundle.

The first connector has can have a maximum cross-sectional dimension of 2.5 mm or less.

The intrauterine access systems can also include a dilator member adapted for sliding over the elongate introducer sleeve.

The present disclosure includes intrauterine access methods. For example, such a method can include providing an optical guidewire having a proximal handle coupled to an elongate shaft configured with a flexible distal shaft portion that carries an image sensor and a light emitter; and introducing a distal end of the optical guidewire trans-cervically into a patient's uterine cavity with endoscopic viewing provided by the image sensor; and advancing a dilator sheath over the optical guidewire trans-cervically to thereby dilate a cervix of a patient. Variations include the elongate optical guide member having a maximum cross-sectional dimension of 2.5 mm or less.

Variations of the method can include introducing the distal end of the optical guidewire trans-cervically by illuminating a trans-cervical path of the optical guidewire with a light emitter. In additional variations, introducing the distal end of the optical guidewire trans-cervically further includes providing a fluid flow into the trans-cervical path of the optical guidewire. The dilator sheath can have an outer diameter of at least 4.0 mm for dilating the cervix of the patient.

In additional variations, introducing the distal end of the optical guidewire trans-cervically can include using an articulating mechanism to articulate the distal end.

Overall, the methods and devices described herein provide safer, more efficient, and patient-centered approach to cervical dilation for various gynecological procedures requiring intrauterine access. The combination of visualization with the optical guidewire and controlled dilation with the dilator offers significant advantages over existing methods.

### BRIEF DESCRIPTION OF THE DRAWINGS

FIG. 1 illustrates a perspective view of a variation of an intrauterine access system comprising (i) an imaging or optical guidewire de-mated from a handle where the optical guidewire has an elongated shaft with a distal tip carrying an image sensor and an LED and (ii) a de-mated introducer sleeve with a lumen therein for receiving the optical guidewire, with a block diagram showing a controller, fluid source and image display operatively connected to the system.

FIG. 2 is a perspective view of a variation of the optical guidewire of FIG. 1, showing a proximal end with electrical contacts, a middle portion of the guidewire shaft being rigid or semi-rigid, and a distal portion of the guidewire shaft being flexible.

FIG. 3 illustrates an enlarged view of a distal tip of the optical guidewire of FIGS. 1 and 2 showing the dimensions of an image sensor and an LED.

FIG. 4 is a cut-away view of a dilator component of the invention comprising a split dilator sheath that is adapted for slidable translation over the assembly of the introducer sleeve and optical guidewire.

FIG. 5A illustrates an initial step in a method of using the guidewire assembly of FIG. 1 to access a patient's uterine cavity, wherein the distal flexible portion of the optical guidewire is advanced through the cervix and cervical canal under direct vision and irrigation from a fluid source.

FIG. 5B illustrates a subsequent step in the method wherein the introducer sleeve is advanced over the guidewire into the uterine cavity, and the split dilator sheath is positioned over the introducer sleeve.

FIG. 5C illustrates a subsequent step in the method wherein the dilator sleeve is advanced over the introducer sleeve to dilate the cervix and cervical canal.

FIG. 5D illustrates a subsequent step wherein the dilator sheath and introducer sleeve are removed from the dilated cervical canal, and an endoscope is positioned for introduction through the dilated cervical canal.

FIG. 6 is a perspective view of another variation of a split dilator sheath which is configured with an inflatable balloon for dilating the patient's cervix and cervical canal.

FIG. 7 is an enlarged perspective view of the distal tip of another variation of optical guidewire that carries a lumen with a distal outlet for delivery of an irrigation fluid from the distal tip of the optical guidewire.

FIG. 8 is an illustration of a guidewire extension member adapted for coupling to the optical guidewire of FIGS. 1 and 2.

FIG. 9. is a schematic view of an endoscope with a working being advanced over an optical guidewire left in place in a patient's cervical canal.

### DETAILED DESCRIPTION

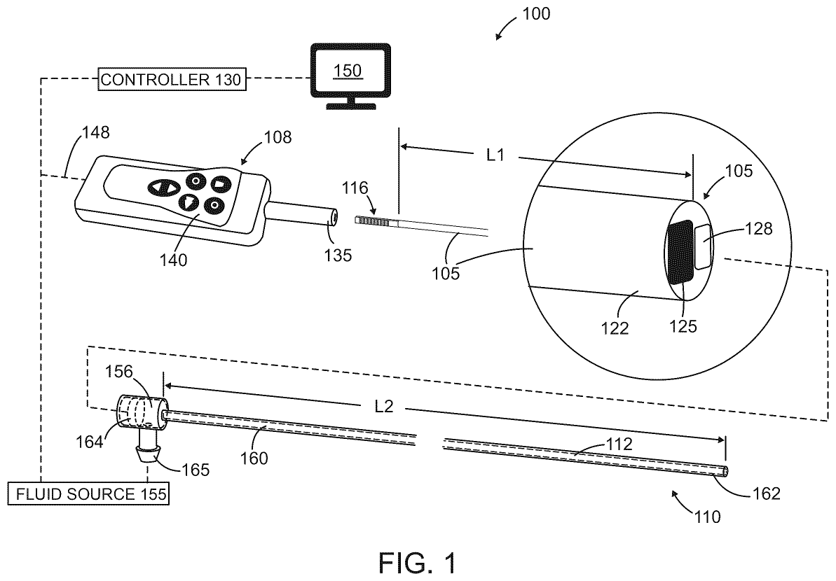

FIG. 1 illustrates a perspective view of a variation of an imaging guidewire system 100 with the components de-mated from one another. The imaging guidewire system 100 comprises an elongate optical guidewire 105 that is detachably coupled to a proximal handle 108. FIG. 1 further illustrates an introducer sleeve 110 with a lumen 112 or passageway therein that is adapted to receive the optical guidewire 105. The assembly of the introducer sleeve 110 and optical guidewire 105 is configured to be advanced through a patient's cervical canal to provide intrauterine access, as described below.

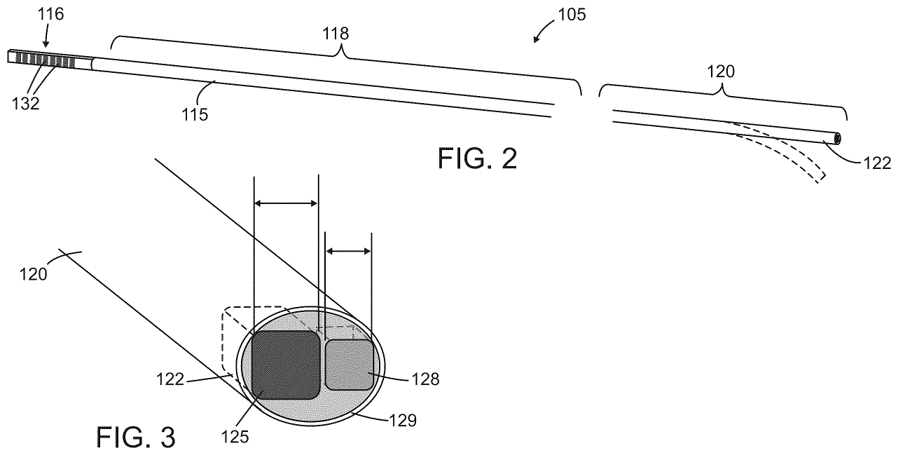

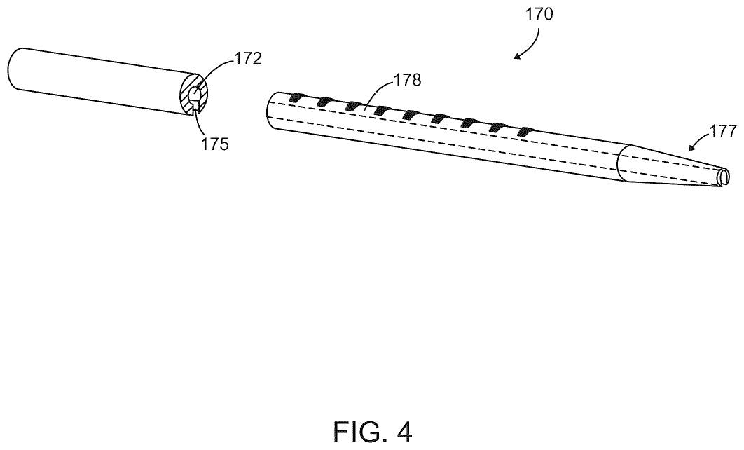

Referring to FIGS. 1, 2, and 3, the optical guide wire 105 comprises a highly elongated shaft 115 with a proximal electrical connector 116, a proximal shaft portion 118, and a distal shaft portion 120 having a distal tip 122 (FIG. 2) that carries an image sensor 125 and a light emitter which can be an LED 128 (FIGS. 1 and 3). The proximal and distal shaft portions 118 120 have an axial length indicated at L1 in FIG. 1.

The guidewire shaft 115 comprises an outer sleeve 129 (FIG. 3) with a passageway therein that carries electrical leads, typically in the form of a flex circuit, for coupling the image sensor 125 and LED 128 to a controller 130 and electrical source therein. The sleeve 129 can be made in part of a biocompatible polymeric material, a biocompatible metallic material, or a combination thereof. In a variation, the sleeve 129, or parts thereof, can be made of a polyolefin, polyethylene (PE), Teflon, a polyamide (e.g., Nylon 6, 11, 12, etc.) or polyether block amide (e.g., PEBAX™), polytetrafluoroethylene (PTFE), polycarbonate (PC), polyetherketone (PEEK), polyethersulfone (PES), polypropylene (PP), polyvinylchloride (PVC), polyvinylidene fluoride (PVDF), perfluoroalkoxy alkane (PFA), or a combination thereof.

As shown in FIG. 2, the electrical connector 116 at the proximal end of the optical guidewire 105 includes a series of electrical contacts 132, for example, of the type of electrical contacts that can be printed on a flexible circuit board and adapted for coupling with a cooperating connector 135 in the handle 108 as shown in FIG. 1. In a variation, the cross-sectional dimension of the electrical connector 116 is the same or similar to the cross-section dimension of the guidewire shaft 115 so that guidewire 105 can be detached from the handle 108 when the guidewire is positioned in the patient's cervical canal and thereafter an endoscope or other surgical instrument can be then advanced over the guidewire 105 without the guidewire being removed from the patient.

Referring again to FIG. 2, the proximal shaft portion 118 of the guidewire shaft 115 can be rigid or a somewhat inflexible polymer sleeve 129 and can include a thin-wall metal tube assembled with the polymer sleeve 129. The distal portion 120 of the guidewire shaft 115 is flexible for the reasons described below and can include a spiral-cut thin-wall metal tube assembled with the polymer sleeve 129. The optical guidewire shaft 115 has an outer diameter or maximum cross-sectional dimension that is less than 2.5 mm and, in a variation, is less than 1.5 mm. In a variation, the cross-sectional dimension is 1.5 mm or less and has an oval cross-sectional shape, as shown in FIG. 3. In a variation shown in FIG. 3, the image sensor 125 has a dimension of 0.5 mm on each side, and the LED 128 has a dimension of approximately 0.4 mm on each side. Another variation that can be used is an LED in size 0201, which is dimensioned 0.3 mm by 0.6 mm. In a variation, the image sensor 125 is a commercially available sensor such as an Omnivision OCHTA10 CMOS sensor (https://www.ovt.com/products/ochta10/), or alternatively an Omnivision sensor OV6946 or Omnivision sensor OV6948. In this variation, the maximum cross-sectional dimension of the distal tip 122 of the optical guidewire 105 is approximately 1.0 mm. In another variation with some image sensors, the distal tip is oval with dimensions of approximately 1.2 mm by 0.8 mm. In a variation, the optical guidewire 115 is sterilizable for multiple uses due to the cost of image sensors, but such an optical guidewire can be configured for limited multiple use or single-use and disposable. In another variation, the entire guidewire shaft 115 can be flexible, and a proximal portion can be stiffened by an external metal sleeve or a stiff polymer sleeve.

In another variation, the light emitting mechanism in the optical guidewire 105 can comprise high acceptance angle glass fibers (35-50 um fibers) with an LED light source in the handle 108 adapted to illuminate the light fibers (not shown).

Referring to FIG. 1, it can be seen that the handle 108 has a control pad 140 with buttons adapted to operate the image sensor 125 and LED 128, for example, to record an image or video or to control light intensity from the LED 128. The handle 108 is coupled to the controller 130 by cable 148 which is further connected to image display 150 for displaying images from the image sensor 125. Also, other operating parameters can be displayed on the image display 150. The control pad 140 also has an actuator button coupled to the controller 130 to operate a pump (not shown) to provide an inflow of an irrigation fluid from fluid source 155, as will be described below.

Still referring to FIG. 1, it can be seen that the introducer sleeve 110 has a proximal hub 156 coupled to elongate sleeve portion 160, extending length L2 to a distal end 162 of the sleeve portion 160. The sleeve portion 160 is typically a biocompatible metallic material such as stainless steel and is rigid but can be made in part of a biocompatible polymeric material or a combination of polymeric and metallic material to provide a sleeve portion 160 that is rigid or substantially rigid. The hub 156 has a slit seal 164, as is known in the art for slidable sealing over the guidewire shaft 115 after the guidewire is inserted through lumen 112 in the introducer sleeve 110. The hub 156 carries a fitting 165 for coupling to fluid inflow tubing 166, which is, in turn, coupled to the fluid source 155 described above. The fluid source 155 provides a fluid flow through the lumen 112 in the introducer sleeve 110. The dimension of lumen 112 in introducer sleeve 110 is slightly larger than the cross-sectional dimension of the guidewire shaft 115 to provide an annular space for fluid flows through the length of introducer sleeve lumen 112 to irrigate the patient's cervix and cervical canal during advancement of the distal portion 120 of the guidewire 105 through the cervix. A continuous fluid flow or pulsed fluid flow can be provided by actuation of one or more buttons on the control pad 140 in the handle 108 by the physician. The axial length L2 of the sleeve portion 160 is at least 5 mm or at least 10 mm less than the axial length L1 of the optical guidewire shaft 115, as described above. The axial length L2 of the introducer sleeve 110 thus allows the physician to extend the flexible distal portion 120 of the optical guidewire 105 a selected distance distally outward from the distal end 162 of the more rigid introducer sleeve 110 for navigating through the cervical canal as will be described below.

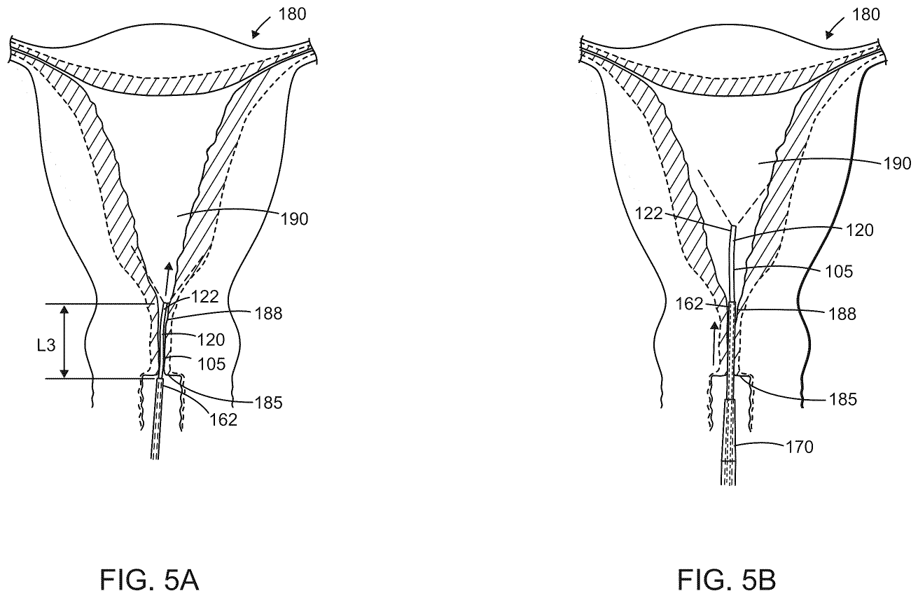

From FIG. 1, it can be understood that the assembly of the optical guidewire 105 and introducer sleeve 110 will allow for visual imaging while introducing the small diameter guidewire tip 122 through a patient's cervix and cervical canal. A purpose of such endoscopic access with the guidewire 105 is to thereafter dilate the cervix to allow for introduction of a larger diameter endoscope with a working channel to then introduce a tool into the uterine cavity to perform a procedure. Thus, FIG. 4 illustrates a variation of a dilator sheath 170 that is configured for advancing over the introducer sleeve 110 to dilate the cervix and cervical canal. The dilator sheath 170 comprises an elongate, somewhat flexible, polymeric member with an axial passageway 172 therein and an axial slit 175 or a helical slit that allows the sheath 170 to be placed over the introducer sleeve 110 and then advanced distally over the introducer sleeve 110 into and through the cervical canal to thereby dilate the canal. The dilator sheath typically has a tapered distal tip 177 and a longer central portion 178 with a suitable cross-sectional dimension, for example, 4 mm, 5 mm, 6 mm, 8 mm, or more. In another variation, several dilator sheaths of varying diameters can be used sequentially to dilate the cervix and cervical canal. In another variation described below, a balloon can be used as a dilation mechanism.

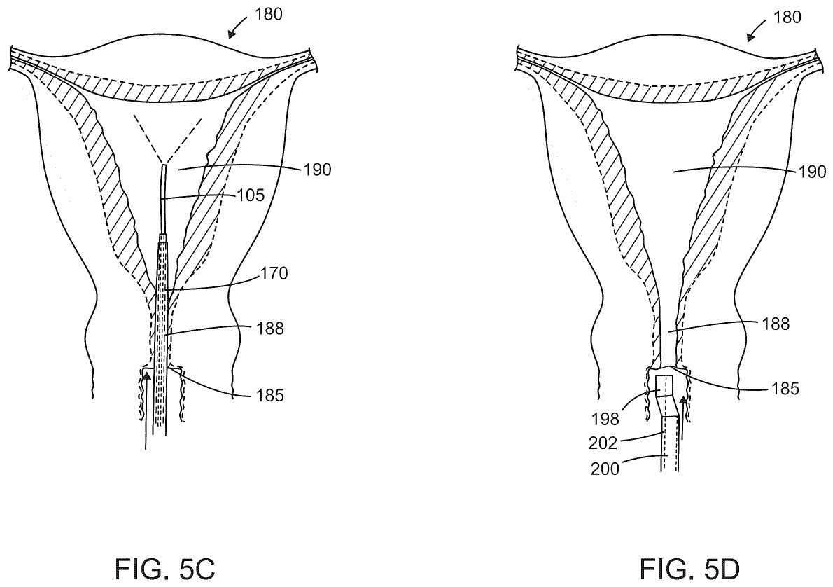

Now, turning to FIG. 5A, a first step in the method of use corresponding to the invention, is shown. FIG. 5A illustrates a patient's uterus 180 with cervix 185, cervical canal 188, and uterine cavity 190. In FIG. 5A, the physician has positioned a selected extended length L3 of the flexible distal portion 120 of the optical guidewire 105 distally from the distal end 162 of the more rigid introducer sleeve 110. This extended length L3 can range from 5 mm to 10 mm or more and can be adjusted during use as the introducer sleeve 110 is slidable over the optical guidewire 105. For example, the extended length L3 of the distal portion 120 can be less when initially inserting the guidewire tip 122 through the cervix 185, so the guidewire tip 122 does not deflect significantly, allowing its insertion through the cervix 185. Thereafter, the extended length L3 of the distal portion 120 can be increased to provide increased flexibility in the extended length L3 to allow the tip 122 to follow the path of least resistance through the cervical canal 188. The increased flexibility of the longer extended length L3 helps to ensure that the tip 122 of guidewire 105 does not penetrate or perforate the wall of the cervical canal 188. The physician typically actuates the fluid source 155 to irrigate the cervix 185 and canal 188 during this step of the method. In a variation, the fluid in fluid source 155 is a saline solution. For example, the fluid can be 0.90% sodium chloride (NaCl) solution. Alternatively, the fluid can be a 0.45% NaCl solution or a solution comprising between about 0.45% to about 0.90% NaCl.

FIG. 5B illustrates a subsequent step in the method wherein after the guidewire tip 122 has entered the uterine cavity 190, the physician advances the assembly of the introducer sleeve 110 and guidewire 105 into the uterine cavity 188. Alternatively, the physician can advance the introducer sleeve 110 over a stabilized guidewire 105. FIG. 5B further shows that the split dilator sheath 170 has been positioned over the introducer sleeve 110 in preparation for advancement and dilation of the cervix 185 and cervical canal 188. The physician again typically actuates fluid source 155 to irrigate the cervix 185 and canal 188 during this step of the method.

FIG. 5C illustrates a subsequent step of the method wherein the physician advances the dilator sheath 170 through the cervix 185 and cervical canal 188 to thereby dilate the cervix and canal.

FIG. 5D illustrates a subsequent step wherein the assembly of the optical guidewire 105 and introducer sleeve 110 have been removed, showing the dilated cervix 185 and cervical canal 188. FIG. 5D further shows the physician positioning the distal end 198 of a larger endoscope 200 with a working channel 202 near the dilated cervix 185 in preparation for advancing the endoscope through the dilated cervix 185 and cervical canal 188. The distal end 198 of the endoscope is illustrated in FIG. 5D is of the type disclosed in commonly-owned U.S. Pat. Nos. 11,937,787, 11,596,298, 11,096,560, 11,529,048, 11,089,951, 11,369,253, 11,259,695, 10,432,717, 11,589,736, 11,019,987, 11,432,717, 11,717,141, 11,832,786 and 11,304,594.

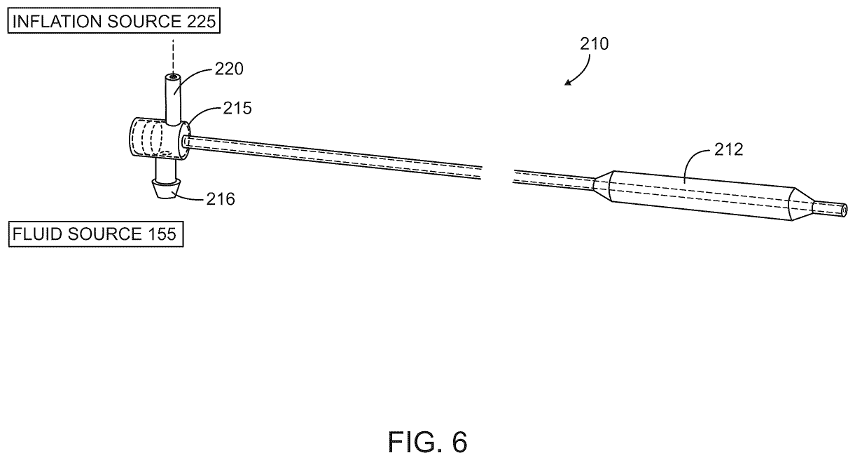

FIG. 6 shows another variation of an introducer sleeve 210 that carries a balloon 212 for dilating a patient's cervix 185 and cervical canal 188. The introducer sleeve 210 has a proximal hub 215 that again carries a fitting 216 for coupling to fluid source 155. The hub 215 further carries a second fitting 220 adapted for coupling to an inflation source 225, such as a syringe. In this variation, the physician advances the optical guidewire 105 through the cervix 185 as described above and thereafter advances the introducer sleeve 210 and balloon 212 into the cervix 185 and cervical canal 188. As a final step, the physician inflates the balloon 212 to dilate the cervix 185 and the cervical canal 188. Thereafter, the physician can introduce a larger working endoscope through the dilated cervix as described above.

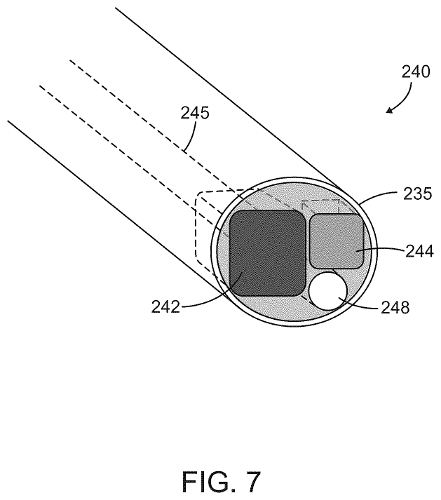

FIG. 7 illustrates the distal end 235 of another variation of an optical guidewire 240, which again carries an image sensor 242 and LED 244 and further includes a lumen 245 for introducing irrigation fluid directly through outlet 248 in the distal end 235 of the guidewire. It can be understood that the hub of the after guidewire can carry the fitting for coupling to a fluid source. This variation is suited for use with an inexpensive image sensor in the future that allows for single use rather than sterilization. In this variation, it may be difficult to sterilize the elongated fluid delivery lumen 245.

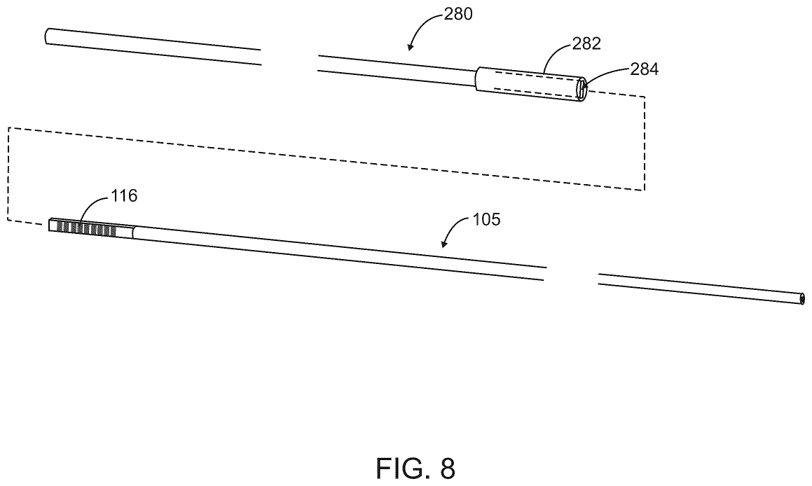

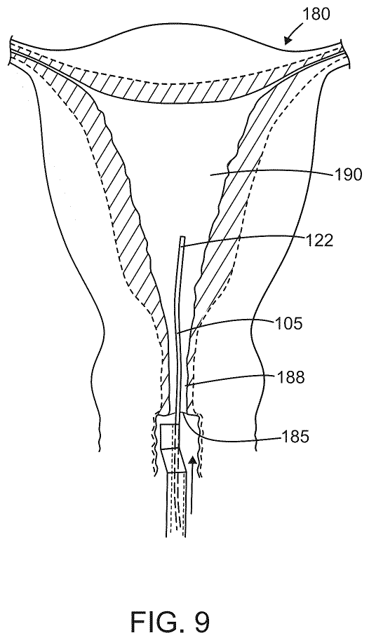

FIG. 8 illustrates another variation of the apparatus and method of the invention that comprises a guidewire extension member 280 that is adapted for coupling to the original guidewire 105 of FIGS. 1 and 2 above. The purpose of the guidewire extension member 280 is to provide a further elongated guidewire assembly that allows for the introduction of an endoscope with a working channel over the guidewire assembly. The assembly of the original optical guidewire 105 and extension member 280 further ensures that introduction of a larger diameter endoscope does not lacerate or perforate the wall of a cervical canal 188. In FIG. 8, it can be seen that the guidewire extension number 280 has a distal end 282 with a recess 284 for detachable coupling with the connector 116 on the original guidewire 105. In this variation, the connector 116 is simply used for a mechanical connection and not an electrical connection. Further, guidewire extension member 280 can have a larger diameter, for example, 1.5 mm to 4.0 mm 2 since the working channel of an endoscope typically ranges from 2 mm to 5 mm. FIG. 9 illustrates a method of using the guidewire system that allows the original guidewire 105 to remain in place, extending through the cervix 185 and cervical canal 188 into the uterine cavity 190 for guiding an endoscope over the guidewire for intrauterine access with a reduced risk of laceration or perforation.

A number of embodiments have been described. Nevertheless, it will be understood by one of ordinary skill in the art that various modifications may be made without departing from the spirit and scope of the embodiments. Methods recited herein may be carried out in any order of the recited events that is logically possible, as well as the recited order of events. Moreover, additional steps or operations may be provided, or steps or operations may be eliminated to achieve the desired result.

Each of the individual variations or embodiments described and illustrated herein has discrete components and features that may be readily separated from or combined with the features of any of the other variations or embodiments. Modifications may be made to adapt a particular situation, material, composition of matter, process, process act(s), or step(s) to the objective(s), spirit, or scope of the present invention.

All existing subject matter mentioned herein (e.g., publications, patents, and patent applications) is incorporated by reference herein in its entirety except insofar as the subject matter may conflict with that of the present invention (in which case what is present herein shall prevail). The referenced items are provided solely for their disclosure prior to the filing date of the present application. Nothing herein is to be construed as an admission that the present invention is not entitled to antedate such material by virtue of prior invention.

This disclosure is not intended to be limited to the scope of the particular forms set forth but is intended to cover alternatives, modifications, and equivalents of the variations or embodiments described herein. Further, the scope of the disclosure fully encompasses other variations or embodiments that may become obvious to those skilled in the art in view of this disclosure.

What is claimed is:

1. An intrauterine access system, comprising;
a handle;
an elongate optical guide member extending about an axis having a maximum cross-sectional dimension of 2.5 mm or less with a flexible distal tip portion carrying an image sensor and a light emitter;
a first connector with electrical contacts at a proximal end of the elongate optical guide member for coupling to a cooperating second connector in the handle;
an elongate introducer sleeve with an interior passageway dimensioned for receiving the elongate optical guide member; and
a hub at the proximal end of the elongate introducer sleeve with a fitting adapted for coupling to a fluid source for delivering a fluid flow into the interior passageway of the elongate introducer sleeve.

2. The intrauterine access system of claim 1, wherein the flexible distal tip portion has an axial length of at least 5 mm.

3. The intrauterine access system of claim 1, wherein the flexible distal tip portion has an axial length of at least 10 mm.

4. The intrauterine access system of claim 1, wherein the light emitter comprises at least one LED.

5. The intrauterine access system of claim 1, wherein the light emitter comprises a fiber optic bundle.

6. The intrauterine access system of claim 1, wherein the first connector has a maximum cross-sectional dimension of 2.5 mm or less.

7. The intrauterine access system of claim 1, further comprising a dilator member adapted for sliding over the elongate introducer sleeve.

8. An intrauterine access method, comprising:
providing an optical guidewire having a proximal handle coupled to an elongate shaft having a diameter of 2.5 mm of less configured with a flexible distal shaft portion that carries an image sensor and a light emitter; and
introducing a distal end of the optical guidewire trans-cervically into a patient's uterine cavity with endoscopic viewing provided by the image sensor; and
advancing a dilator sheath over the optical guidewire trans-cervically to thereby dilate a cervix of a patient.

9. The intrauterine access method of claim 8, wherein introducing the distal end of the optical guidewire trans-cervically further comprises illuminating a trans-cervical path of the optical guidewire with a light emitter.

10. The intrauterine access method of claim 9, wherein introducing the distal end of the optical guidewire trans-cervically further includes providing a fluid flow into the trans-cervical path of the optical guidewire.

11. The intrauterine access method of claim 8, wherein the dilator sheath has an outer diameter of at least 4.0 mm for dilating the cervix of the patient.

12. The intrauterine access method of claim 8, wherein introducing the distal end of the optical guidewire trans-cervically includes using an articulating mechanism to articulate the distal end.
